(19) United States
(12) Patent Application Publication
(10) Pub. No.: US 2007/0129701 A1
(43) Pub. Date: Jun. 7, 2007

(54) Method And Apparatus For Providing A Garment

(76) Inventor: Marcella Leone, Stamford, CT (US)

Correspondence Address: ST. ONGE STEWARD JOHNSTON & REENS, LLC, 986 BEDFORD STREET, STAMFORD, CT, 06905-5619, US

(21) Appl. No.: 11/559,744

(22) Filed: Nov. 14, 2006

Related U.S. Application Data
(60) Provisional application No. 60/736,196, filed on Nov. 14, 2005.

(51) Int. Cl. A61F 13/15

(52) U.S. Cl. 604/387

(57) ABSTRACT

The invention relates to a method and apparatus for providing a garment that is secured to a first arm and a second arm of a wearer, including the steps of providing a sheet of material with a first connector attached to one side of the sheet and a second connector attached to an opposite side of the sheet, placing the first arm through the first connector, and extending the sheet over a first breast proximate the first arm and under the second arm. The method also includes extending the sheet across a back of the wearer and under the first arm of the wearer, extending the sheet across a front of the wearer and over a second breast proximate the second arm, and placing the second arm through the second connector.

### CROSS-REFERENCE TO RELATED APPLICATIONS

This patent application claims the benefit of, under Title 35, United States Code, Section 119(e), U.S. Provisional Patent Application No. 60/736,196, filed Nov. 14, 2005.

### FIELD OF THE INVENTION

The invention relates to a garment that can be adjusted to fit various wearers regardless of their particular size, shape, or weight.

### BACKGROUND OF THE INVENTION

Adjustable garments are typically provided to fit a wearer as the size of the wearer changes and for reducing the amount of non-adjustable garments a wearer purchases to accommodate a changing figure. In addition, an adjustable garment may reduce the amount of sewing needed to take in or let out a non-adjustable garment in the event the wearer does not purchase multiple garments in multiple sizes.

Therefore, an adjustable garment often reduces expenses because the wearer may not need to purchase and maintain multiple garments in multiple sizes and an adjustable garment usually saves time and burden by reducing efforts for taking in or letting out the garment that has become too small or too big for a wearer. Another possible disadvantage of having numerous non-adjustable garments is that they take up space in a home, and closet space is often a premium in most homes.

U.S. Pat. No. 2,259,236 to Wellins seems to show a maternity shirt or dress which is adjustable to accommodate varying sizes of the waist of the wearer.

U.S. Pat. No. 898,511 to Scheider appears to disclose a shirt having a means for gathering the skirt at the back for changing the size of the waist so that the skirt may be fitted to different figures. U.S. Pat. No. 2,394,019 to Terrell relates to a draw string in the form of a gathered waist belt so that the waist size can be readily altered to suit the desires of the wearer in a manner whereby the waist part of the garment will at all times retain a pleasing appearance.

Another type of adjustable skirt is disclosed in U.S. Pat. No. 723,063 to Stillson wherein tapes are typically employed on either side of the placket opening to permit adjustment to fit any size or shape of waist or hips. After adjustment, the tapes are tied to maintain the adjustment irrespective of the nature or use of the placket fastening devices.

U.S. Pat. No. 2,418,582 to Stumpf touches upon a simple garment in which fit is gained through the use of a plurality of drawstrings which are located to shape the garment at the points required. U.S. Pat. No. 2,327,881 to Friedman generally relates to the use of a two piece waistline belt which is adapted to be used in conjunction with a uniform having a bodice portion and a skirt portion in manner to cause the skirt portion to drape in an outwardly flared formation. U.S. Pat. No. 2,109,867 to Pedlar typically relates to a combination garment comprising a blouse and a petticoat wherein the upper edge of the petticoat is attached to the lower edge of the blouse by employing a slide fastener.

U.S. Pat. No. 4,377,007 to Sudjian generally disclosed a convertible length garment having an overdress and a complimental under dress which is adapted to be worn beneath the overdress.

Although all of the above prior references relate to adjustable garments, the time and effort needed to make the adjustments may be difficult or cumbersome, in which case the wearer may be discouraged from making the adjustment. Moreover, if the adjustable garment requires a significant amount of time or effort from the wearer to make the adjustment, this also can be discouraging to the wearer. Further, even if the adjustment is easy to perform in little time, the adjustment should be effective so that the wearer need not repeat the adjustment or fix the tension of the garment because the adjustment is too tight or too lose. The use of a draw string adjustment, as mentioned above in several references, is often quick but not effective as the draw string may loosen, in which case the wearer needs to redo the adjustment, or be too tight or too lose, in which case the wearer also needs to redo the adjustment.

What is desired, therefore, is a garment that is easily adjustable to fit a variety of sizes and shapes. Another desire is an adjustable garment that maintains the fit about a wearer after the adjustment. A further desire is an adjustable garment that continues to maintain its fit about the wearer after repeated use adjustment mechanism. Yet another desire is to achieve all of the above advantages without sacrificing ease of use, aesthetic appeal, comfort, or functionality of the garment. Another desire is a garment that appears to slim the figure while maintaining its adjustability and ease of use. A still further desire is a garment that does not require fasteners, closers, and the like.

### SUMMARY OF THE INVENTION

These and other objects of the invention are achieved by a garment that is easy to put on and take off without cumbersome closers or fasteners.

Another object is a garment that is easy to adjust and once adjusted, the adjustment stays in place.

A further object is a garment that can be repeatedly adjusted and achieve consistent fit each time the garment is adjusted.

Yet another object is a garment that fits a plurality of sizes.

These and other objects of the invention are achieved by a method for providing a garment that is secured to a first arm and a second arm of a wearer, including the steps of providing a sheet of material with a first connector attached to one side of the sheet and a second connector attached to an opposite side of the sheet, placing the first arm through the first connector, and extending the sheet over a first breast proximate the first arm and under the second arm. The method also includes extending the sheet across a back of the wearer and under the first arm of the wearer, extending the sheet across a front of the wearer and over a second breast proximate the second arm, and placing the second arm through the second connector.

In some embodiments, the method adjusts a length of the first connector. In other embodiments, the method adjusts a width of the sheet as the sheet extends across the back of the wearer. Optionally, the method permits adjustment of the vertical location of the sheet as the sheet extends across the back of the wearer.

The method also provides a reversible sheet of material where the wearer selects either side to face outwardly. Another embodiment includes cinching the waist with a belt attached to the garment.

In further embodiments, the method includes the step of extending the sheet under the second breast proximate the second arm after the step of extending the sheet over the first breast proximate the first arm and before the step of extending the sheet under the second arm. In another embodiment, the method includes the step of extending the sheet under the first breast proximate the first arm after the step of extending the sheet under the first arm of the wearer and before the step of extending the sheet over the second breast.

In all of the above embodiments, the method secures the garment to the wearer without using a fastener to secure the garment to the wearer.

In another aspect of the invention, a garment that is secured to a first arm and a second arm of a wearer includes a sheet of material for wrapping about the wearer, where the sheet is sized for encompassing the wearer more than one time. The sheet also includes a first connector attached to one side of the sheet for accommodating the first arm and a second connector attached to an opposite side of the sheet for accommodating the second arm. The sheet also includes a left piece and a right piece where the left piece is attached to the right piece in one of a plurality of locations. Each connector has a length that is adjustable to one of a plurality of adjustments.

In some embodiments, the garment has a second sheet for wrapping about the wearer. In further embodiments, a third connector is attached to the one side for accommodating the first arm and a fourth connector is attached to the opposite side for accommodating the second arm.

It is understood the first and second connectors secure the garment to the wearer without a fastener, such as a button, a zipper, an adhesive, and combinations thereof.

### BRIEF DESCRIPTION OF THE DRAWINGS

FIGS. 1a-1d depict the method for providing the garment in accordance with the invention.

FIG. 2 depicts a flowchart for providing the method shown in FIG. 1.

FIG. 3 depicts the garment shown in FIG. 1 with long sleeves.

FIG. 4 depicts the garment shown in FIG. 1 with short sleeves.

FIGS. 5a-5c depict variations of the connector shown in FIG. 1.

FIGS. 6a-6d depict variations of the sheet shown in FIG. 1.

FIG. 7 depicts a modification to the garment shown in FIG. 1.

FIG. 8 depicts another modification to the garment shown in FIG. 1.

FIG. 9 depicts another modification to the garment shown in FIG. 1.

FIG. 10 depicts a layered look of the garment shown in FIG. 1.

FIGS. 11a-11d depict adjustments to a back of the garment shown in FIG. 1.

### DETAILED DESCRIPTION OF THE INVENTION

As shown in all the figures, garment 20 is a dress comprising sheet 22 of material that is generally flat with first and second arm connectors 22, 24. Each connector has the same limitations as the other and, in some places and for purposes of simplicity, first connector 22 is described but it is understood that second connector 24 includes the same limitations as described for first connector 22.

As shown in all figures, garment 20 is wrapped about the user and provides a one size fit all type of fit since the wrapping is done around the user to the user's desire for snugness, appearance, and comfort. Since garment 20 is wrapped about the wearer, it gives a slimmer appearance to the wearer. Moreover, garment 20 permits customization since it is wrapped about the user according to the user's preferences for fit.

Still further, garment 20 is quick to put on and does not use fasteners or closers, such as buttons or zippers or adhesives, to secure the dress onto the wearer. Without buttons, there is no broken or lost buttons needing to be replaced. Garment 20 also does not require the wearer to place it over the head, which may negatively affect any hair style that is in place. Further, because garment 20 wrapped about the user, it appears to give the user a slimmer appearance.

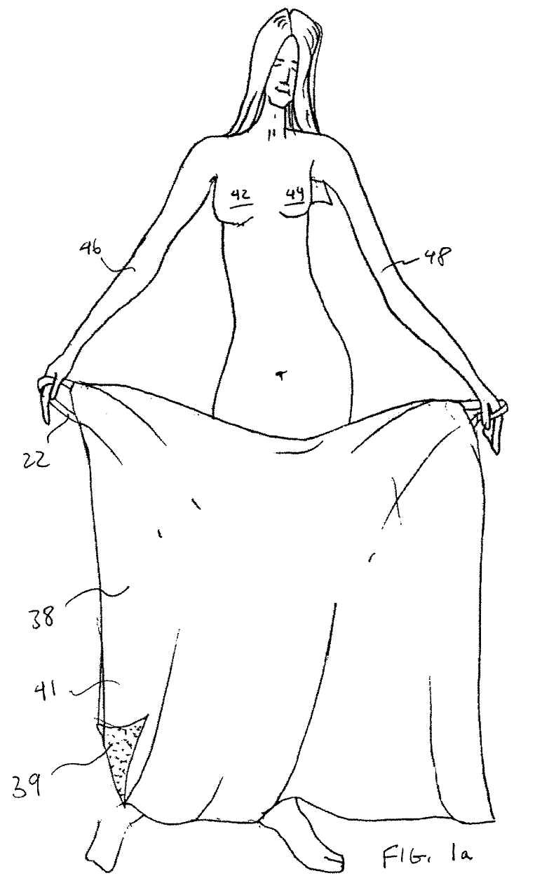

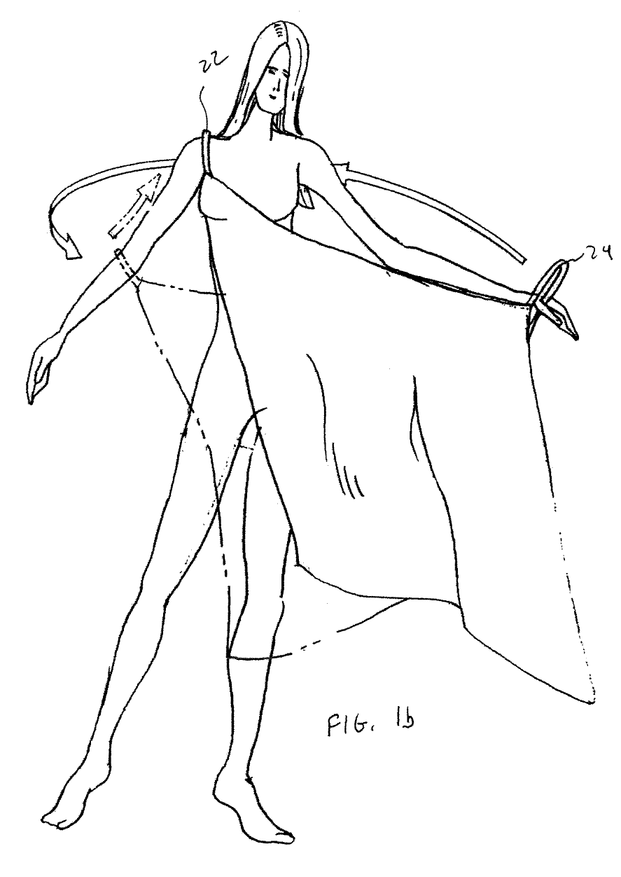

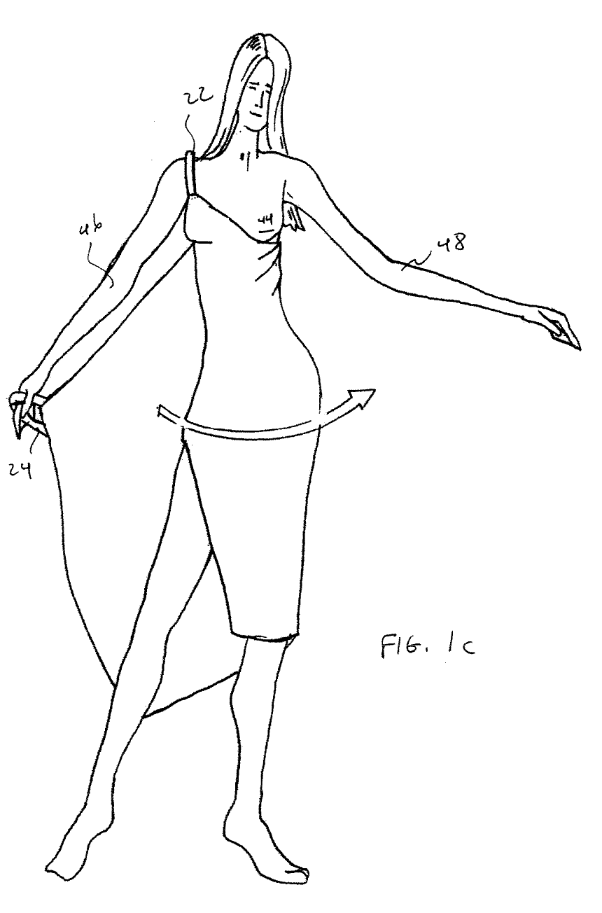

As shown in FIGS. 1a-1d, garment 20 functions by a wearer placing first arm 46 through first connector 22, extending sheet 38 over first breast 42 of the wearer and then under second breast 44, and extending sheet 38 under second arm 48 toward a back of the wearer.

Next, sheet 38 is extended across a back of the wearer and under first arm 46. Garment further functions by extending sheet 38 across a front of the wearer again but this time under first breast 42 and over second breast 44. The wearer then passes second arm 48 through second connector 24.

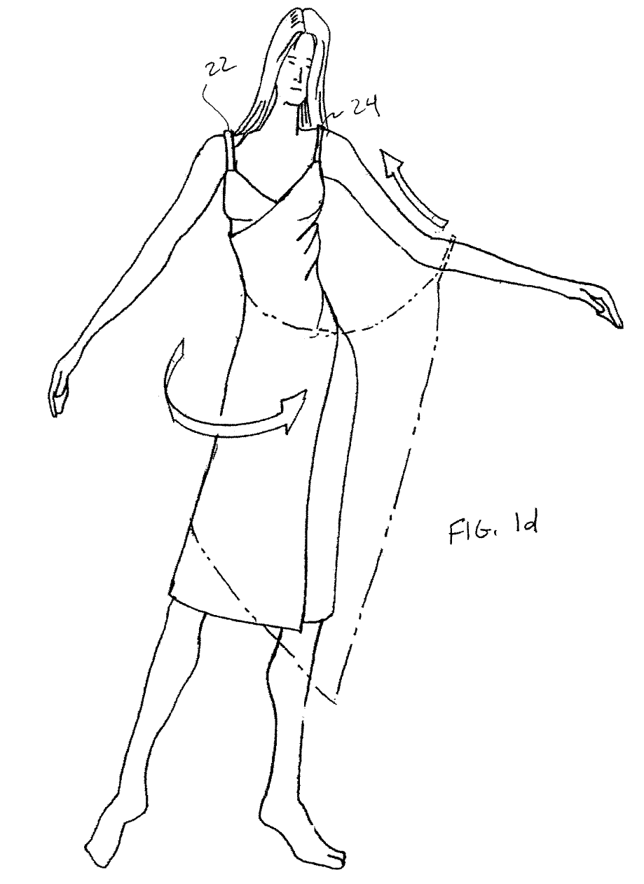

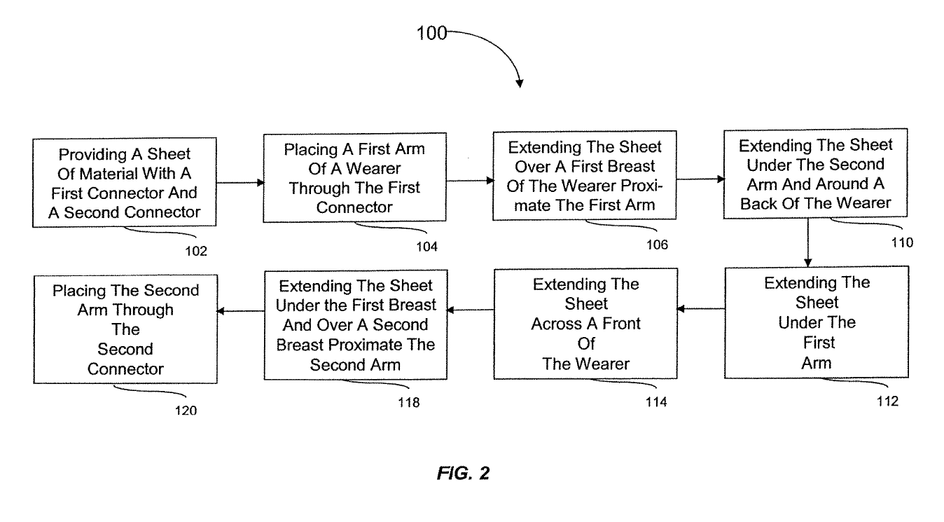

As shown in FIG. 2, method 100 for providing garment 20 is shown, including the steps of providing 102 a sheet of material with a first connector and a second connector, placing 104 the first arm of the wearer through the first connector, and extending 106 the sheet over a first breast proximate the first arm and under the second breast proximate the second arm.

Method 100 also includes extending 110 the sheet under the second arm and around a back of the wearer, extending 112 the sheet under the first arm, and extending 114 the sheet across a front of the wearer.

Method 100 also includes extending 118 the sheet under the first breast and over the second breast proximate the second arm and placing 120 the second arm through the second connector. As shown, garment 20 is reversible where sheet 38 may be worn, at the wearer's choice, where either side one 39 or side two 41 may be facing inwardly toward the wearer or outwardly away from the wearer.

Garment 20 is reversible and includes two sides of different materials, such as terry cloth material on one side and a lycra or water repellant material on the other side. Hence, both side one 39 and side two 41 may be worn inwardly or outwardly, alternatively from one another. In some of these embodiments, the two sides having varying decorative features, such as different colors, indicia, or ornaments. Terry cloth and lycra are not exclusive of the types of material used. In some embodiments, garment 20 includes sequins, spandex, cotton, acrylic, polyester, leather, suede, and the like.

Optionally, garment 20 has wording or indicia printed or sewn on the inside and/or outside of garment 20. A reversible garment permits the wearer to select which indicia to display by flipping garment 20 inside out. Indicia or wording may further include cartoon characters or rhymes or children's songs. Another optional feature of garment 20 is a charm attached to garment 20, where the charm has wording, indicia, and/or logo on it.

In some embodiments (FIGS. 1a-1d), connector 22 is a loop attached to a side of flat material 28. In other embodiments, connector 22 is a loop with sleeves 23 attached, whether long or short sleeves (see FIGS. 3-4). In further embodiments, connector 22 is attached to ruffles or other ornaments. In yet other embodiments, sleeves 23 may be puffed. In an optional embodiment, garment 20 includes belt 33 for cinching a waist of the wearer.

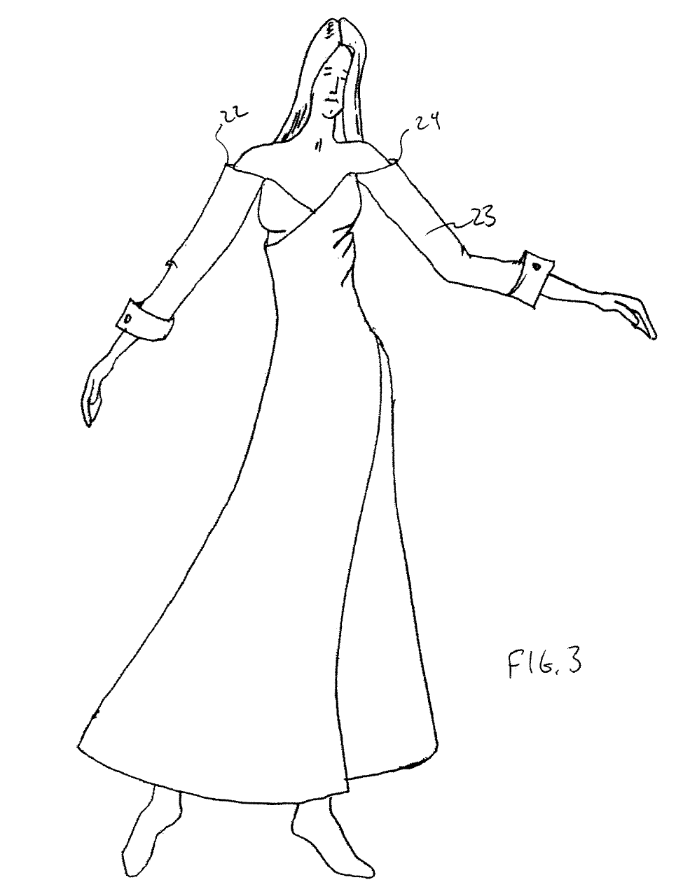

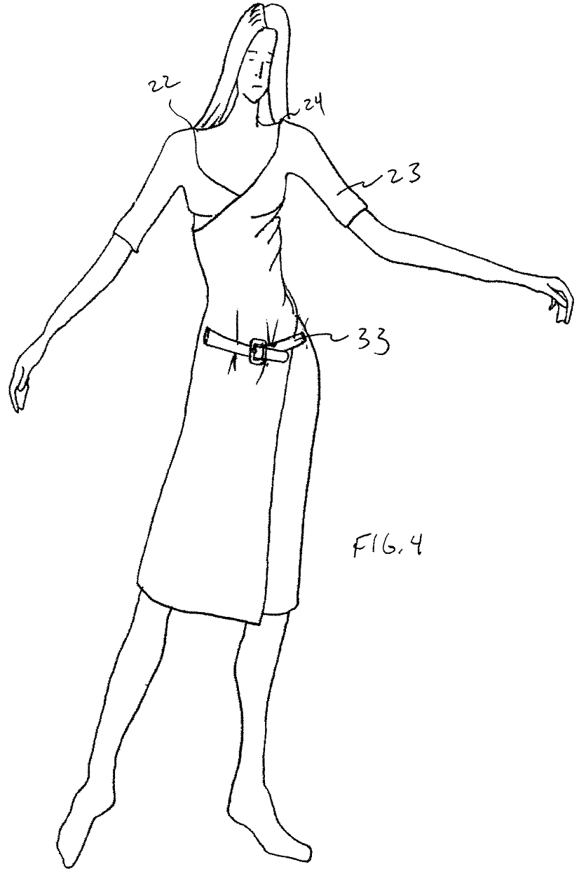

In another embodiment shown in FIG. 5a, adjustment to connector 22 may be made in its length by a belt-like clasp mechanism 32 and where strap 23 has plurality of holes and where a wearer would select one hole for placement of prong 34 of belt-like clasp mechanism 32 to secure adjustment, in which case the attachment of strap 23 to anchoring strap 25 is adjustably fixed.

In another embodiment shown in FIG. 5b, clasp mechanism 32 permits strap 23 to pass through clasp mechanism 32 several times much like a back pack or brassiere permits adjustment to the shoulder strap.

In a further embodiment shown in FIG. 5c, connector 22 has hook and loop fastener 27 for adjusting a length of connector 22.

In combination with or without the adjustments to connector 22 shown in FIGS. 5a-5c, sheet 38 includes adjustment to vary the fit of sheet 38 as it encompasses or wraps about the wearer.

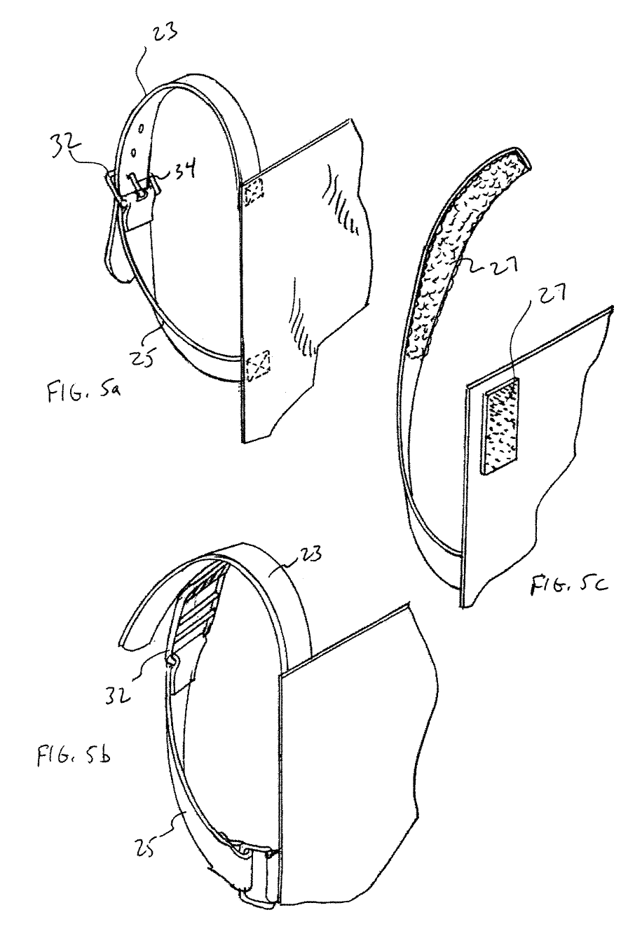

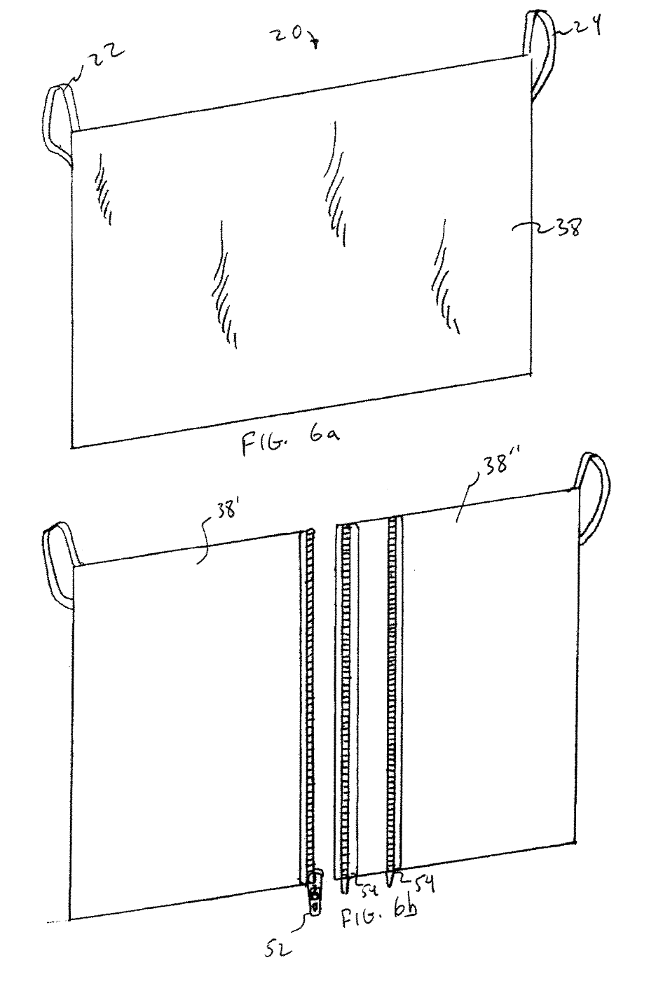

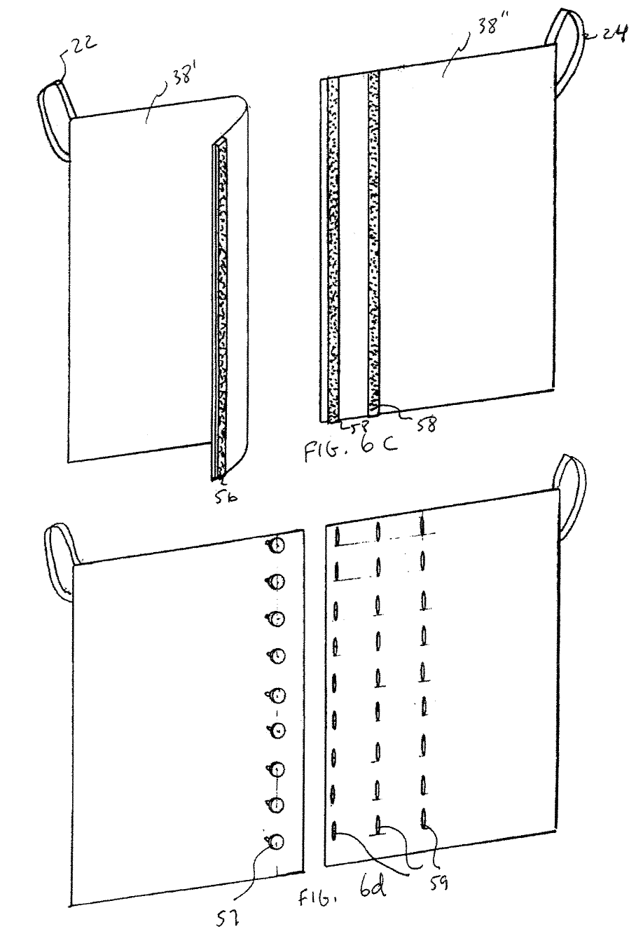

FIG. 6a depicts sheet 38 as it is applied in FIGS 1a-1d. FIG. 6b shows sheet 38 in two pieces defined by left piece 38′ and right piece 38 ″ where the wearer would select a particular fit by attaching first zipper 52 with any one of a plurality of second zippers 54.

FIG. 6c depicts left piece 38′ attached to right piece 38″ in any one of a multitude of locations as determined by the wearer, where the wearer would select a particular fit of garment 20 by attaching hood and loop fastener 56 with complementing hood and loop fastener 58. FIG. 6d achieves the same result using button 57 and apertures 59.

In another embodiment of the invention, garment 20 would be in various lengths to accommodate children or adult wear, including being in a long gown length similar to FIG. 3 or a shorter knee-length dress similar to FIG. 4.

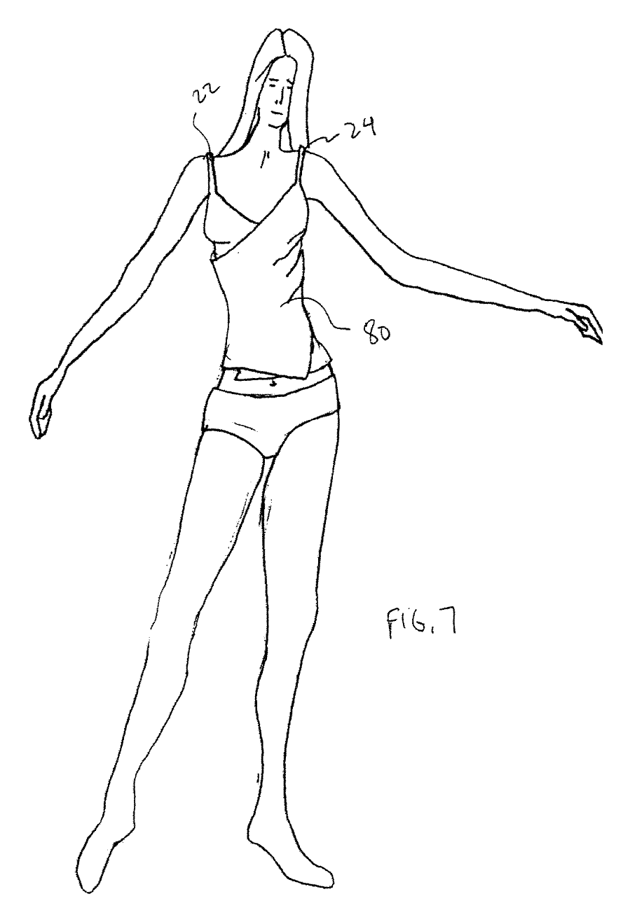

Additionally, as shown in FIG. 7, garment 20 is shirt 80 that utilizes the wrap around feature shown in FIGS. 1a-1d. Shirt 80 includes all the limitations found in garment 20, including variations in size, sleeves, and being sleeveless.

For an expectant mother, shirt 80 is generously cut to accommodate the baby. In some of these embodiments, the generosity of the cut would vary from garment 20 or shirt 80 to accommodate an expectant mother according to trimesters or weeks of gestation.

In another aspect of the invention shown in FIG. 8, an A line style includes sheet 38 of material that would be generally trapezoidal in shape, as opposed to a generally rectangular or square shape. In this aspect, a roomier bottom half of the dress is provided when garment 20 is worn.

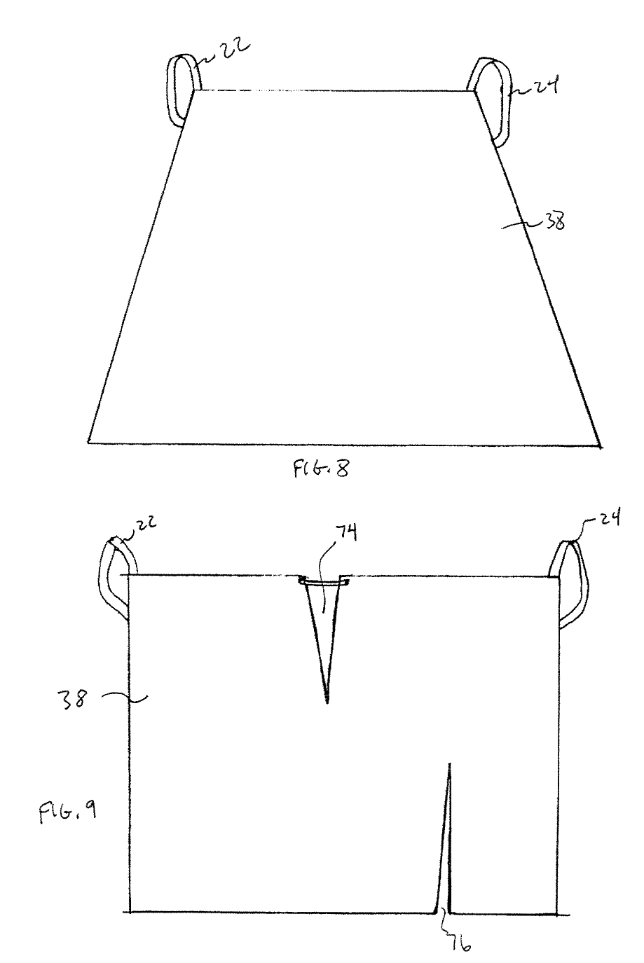

FIG. 9 depicts yet another embodiment where sheet 38 of material 20 includes cut-out 74 such that, when garment 20 is worn by the wearer, a deep V back or a more revealing look of the back of the wearer results. Further, slit 76 in the bottom of sheet 38 would further provide another look by revealing at least one leg of the wearer. Other variations of this embodiment include two slits for revealing both legs.

As an option for any of the embodiments described herein, garment 20 includes a supporting system, such as a brassiere, sewn inside garment 20. This obviates the need for the wearer to wear a brassiere separately.

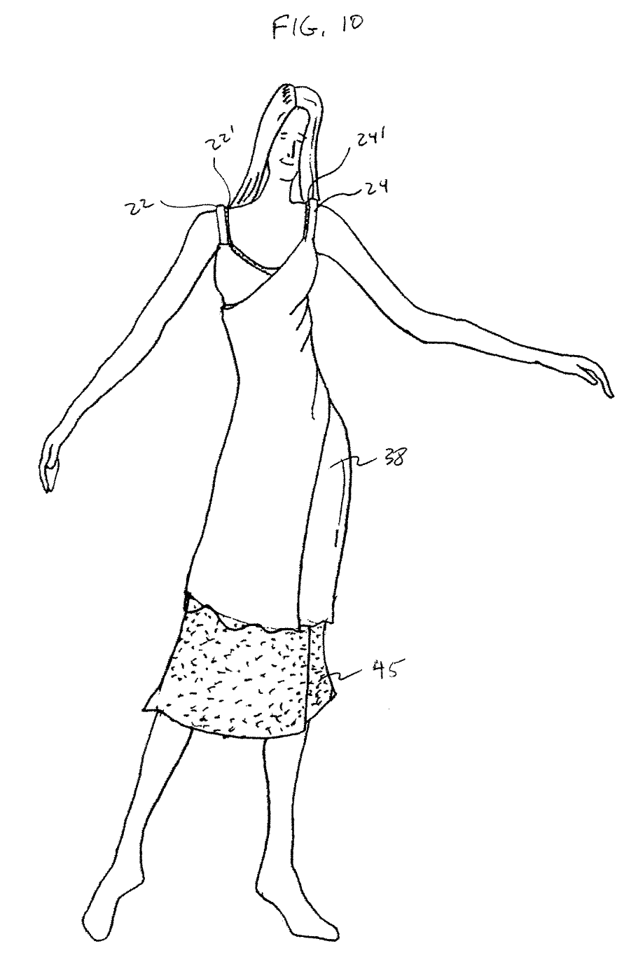

For a layered look shown in FIG. 10, garment 20 includes second sheet 45 that acts as an interior panel which has a length longer than, and therefore extends lower than, sheet 38. Because second sheet 45 is attached to sheet 38, no special assembly or action is needed by the wearer to wear second sheet 45 that is any different from simply wearing sheet 38 and garment 20 as described above. In some of these embodiments, garment 20 includes a layered look, including double connectors 22′ and 24′ that are placed proximate to first and second connectors, 22, 24, respectively.

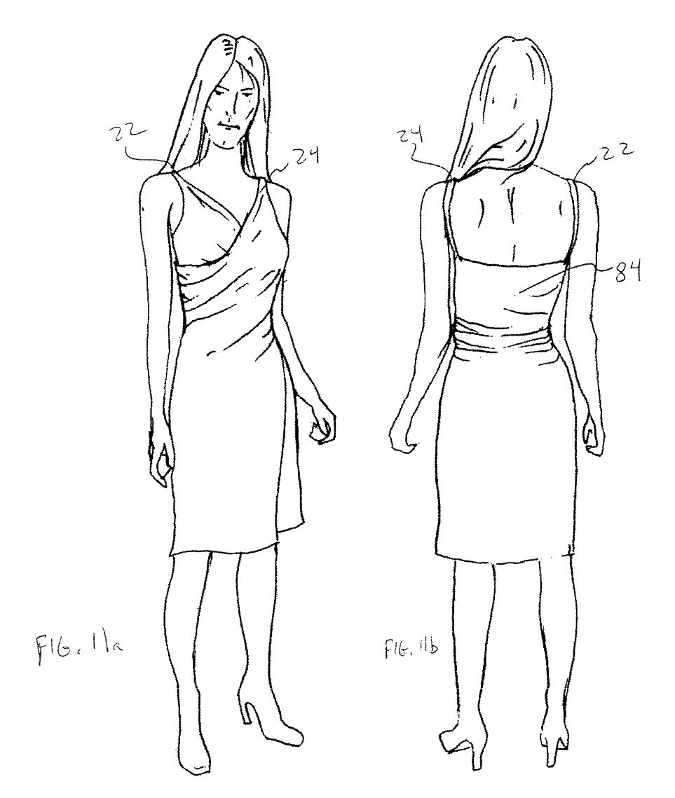

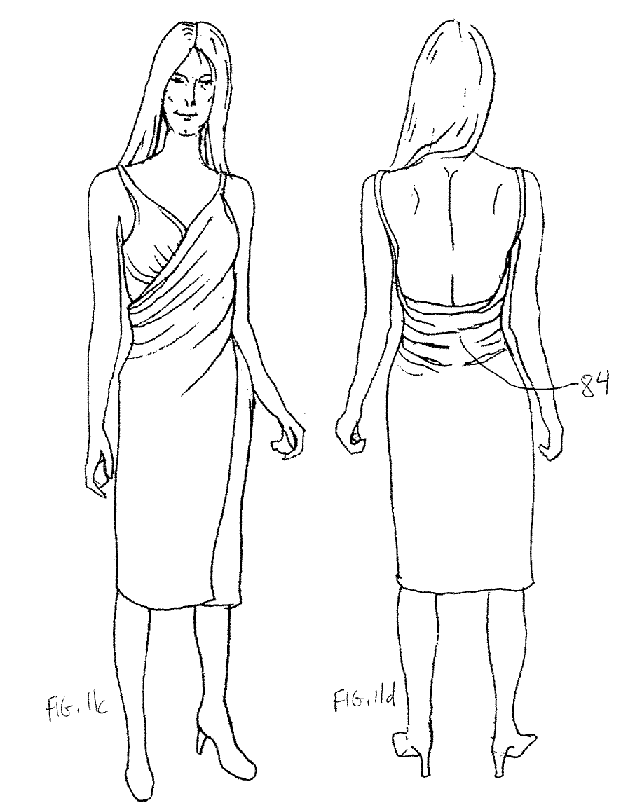

As shown in FIGS. 11a-11d, garment 20 includes adjustable back 84, where the part of sheet 38 that extends around a back of the wearer is adjustable by, due to sheet 38 being an elastic material, simply pulling sheet either downwardly or upwardly. As shown in FIGS. 11a-11b, back 84 is in the up position and, therefore, garment 20 has fewer wrinkles or bunching in a front view. In FIGS. 11c-11d, back 84 is in the down position, resulting in garment having more bunching in the front view. It is understood that the wrinkles or bunching in the front view enhances the wrapping aesthetic appearance and slimming look.

In some of these embodiments, a hook and loop fastener is used to hold back 84 in position to keep back from moving up or down. Alternatively to using a hook and loop fastener, garment 20 may include buttons, adhesive, or any other removably securing mechanism to inhibit back 84 from moving.

Although sheet 38 is described to be an elastic or stretchable material, other embodiments use other materials, such as cotton, polyester, nylon, silk, canvas, woven, unwoven, and the like or combinations of these.

What is claimed is:

1. A method for providing a garment that is secured to a first arm and a second arm of a wearer, comprising the steps of:
providing a sheet of material with a first connector attached to one side of the sheet and a second connector attached to an opposite side of the sheet;
placing the first arm through the first connector;
extending the sheet over a first breast proximate the first arm and under the second arm;
extending the sheet across a back of the wearer and under the first arm of the wearer;
extending the sheet across a front of the wearer and over a second breast proximate the second arm; and
placing the second arm through the second connector.

2. The method according to claim 1, further comprising the step of adjusting a length of the first connector.

3. The method according to claim 1, further comprising the step of adjusting a width of the sheet as the sheet extends across the back of the wearer.

4. The method according to claim 1, further comprising the step of adjusting the vertical location of the sheet as the sheet extends across the back of the wearer.

5. The method according to claim 1, further comprising the step of providing a reversible sheet of material where the wearer selects either side to face outwardly.

6. The method according to claim 1, further comprising the step of cinching the waist with a belt attached to the garment.

7. The method according to claim 1, further comprising the step of extending the sheet under the second breast proximate the second arm after the step of extending the sheet over the first breast proximate the first arm and before the step of extending the sheet under the second arm.

8. The method according to claim 1, further comprising the step of extending the sheet under the first breast proximate the first arm after the step of extending the sheet under the first arm of the wearer and before the step of extending the sheet over the second breast.

9. The method according to claim 1, further comprising the step of securing the garment to the wearer without using a fastener to secure the garment to the wearer.

10. A garment that is secured to a first arm and a second arm of a wearer, comprising:
a sheet of material for wrapping about the wearer;
said sheet is sized for encompassing the wearer more than one time;
a first connector attached to one side of said sheet for accommodating the first arm;
a second connector attached to an opposite side of said sheet for accommodating the second arm;
said sheet includes a left piece and a right piece where said left piece is attached to said right piece in one of a plurality of locations; and
said first and second connectors each has a length that is adjustable to one of a plurality of adjustments.

11. The garment according to claim 10, further comprising a second sheet for wrapping about the wearer.

12. The garment according to claim 10, further comprising a third connector attached to said one side for accommodating said first arm.

13. The garment according to claim 10, further comprising a fourth connector attached to said opposite side for accommodating said second arm.

14. The garment according to claim 10, wherein said first and second connectors secure the garment to the wearer without a fastener.
